(19) United States
(12) Patent Application Publication
(10) Pub. No.: US 2005/0197598 A1
(43) Pub. Date: Sep. 8, 2005

(54) Method and apparatus for taking biomechanical measurements

(76) Inventor: Peter A. Von Rogov, Hillsborough, CA (US)

Correspondence Address: WILSON SONSINI GOODRICH & ROSATI, 650 PAGE MILL ROAD, PALO ALTO, CA, 94304-1050, US

(21) Appl. No.: 11/074,590

(22) Filed: Mar. 7, 2005

Related U.S. Application Data
(60) Provisional application No. 60/550,834, filed on Mar. 5, 2004.

(51) Int. Cl. A61B 8/00

(52) U.S. Cl. 600/587

(57) ABSTRACT

Measurement devices and methods of using them to obtain measurements of a body portion are described herein. One measurement device described includes a base having a rectangular shape including elongated sides and an anatomically shaped end configured to engage with a musculoskeletal anatomical landmark on a body along with a plurality of measurement increments spaced along an elongated side to indicate distance from the anatomically shaped end. One measurement method described includes placing a measurement device having an elongate side and an anatomically shaped end adjacent the body portion to be measured so that the anatomically shaped end engages a musculoskeletal anatomical landmark adjacent the body portion to be measured. The method text includes using measurement increments on the elongate side of the measurement device to determine a position for taking a measurement on the body portion relative to the musculoskeletal anatomical landmark engaged by the anatomically shaped end.

### RELATED APPLICATION

This application claims priority to U.S. Provisional Patent Application Ser. No. 60/550,834 entitled, “Method and Apparatus for Taking Biomechanical Measurements,” filed Mar. 5, 2004 to Peter A. Von Rogov, the entirety of which is incorporated herein by reference.

### BACKGROUND OF THE INVENTION

This invention relates generally to the field of devices for use in making various anatomical and biomechanical measurements involving the limbs, the joints, and appendages of human and nonhuman mammals and, in particular, to measurement devices used to obtain measurements.

Anatomical measurements are an important clinical tool in the evaluation of many disorders. The repeatability of measurements on the same patient over time is critical to obtaining an accurate diagnosis. The reliability of measurements taken among a wider patient population is important for accurate statistical analysis and long-term evaluation of disorders and their potential remedies.

Conventional measurement devices are not configured for cooperative placement against the anatomy being measured. Generally, the measurement is as accurate as the measurer's recollection of the placement of the measurement device on the anatomy. As a result, anatomical measurements taken by conventional measurement devices can produce variable measurement results in the same patient. Because conventional devices are not repeatedly placed the same on every patient, comparisons between large patient populations can suffer from low statistical reliability because of the variability of the individual measurements taken. As a result, anatomical measurements suffer from variability induced not only by variations of interpreting the measurement device, but also from inconsistent or variable placement of the measurement device relative to the anatomy being measured.

In view of the shortcomings and challenges in the use of conventional measurement devices, improved measurement devices and techniques for obtaining reliable and repeatable anatomical measurements are needed.

### SUMMARY OF THE INVENTION

In accordance with an embodiment of the present invention, a measurement device is provided having a base with a rectangular shape including elongated sides and an anatomically shaped end configured to engage with a musculoskeletal anatomical landmark on a body. The measurement device also includes a plurality of measurement increments spaced along an elongated side to indicate distance from the anatomically shaped end.

In accordance with another embodiment of the present invention, a measurement device is provided having a base with an elongated side and an anatomically shaped ends configured to engage with a musculoskeletal anatomical landmark on a body and a plurality of measurement increments spaced along the elongated side. Additionally, an aperture formed within the base at a predetermined measurement increment an aperture is provided within the base at a predetermined increment from the anatomically shaped end.

In accordance with another embodiment of the present invention, a measurement device is provided having a base with an elongated side and an anatomically shaped ends configured to engage with a musculoskeletal anatomical landmark on a body and a plurality of measurement increments spaced along the elongated side to indicate distance from the anatomically shaped end. Additionally, there is at least one aperture within the base at a predetermined measurement increment from the anatomically shaped end and a measurement device is coupled to the base.

In accordance with another embodiment of the present invention, a goniometer is provided having a hub, an arm coupled to the hub and having a rectangular shape include elongated sides and an anatomically shaped end configured to engage with the musculoskeletal anatomical landmark on a body. Additionally, there is provided a plurality of measurement increments spaced along an elongated side of the arm to indicate distance from the anatomically shaped end.

In accordance with additional embodiments of the present invention, there are provided methods for obtaining a measurement on a body portion. In one aspect, the measurement method includes placing a measurement device having an elongate side and an anatomically shaped end adjacent the body portion to be measured so that the anatomically shaped end engages a musculoskeletal anatomical landmark adjacent the body portion to be measured; and using measurement increments on the elongate side of the measurement device to determine a position for taking a measurement on the body portion relative to the musculoskeletal anatomical landmark engaged by the anatomically shaped end. In one specific aspect, the measurement method includes taking a measurement of the body portion at the determined position for taking a measurement on the body portion. In one specific aspect, the measurement comprises a circumference of the body portion. In other specific aspects, the measurement method includes marking the body portion at the determined position for taking a measurement, attaching another measurement device to the elongate side at the determined position for taking a measurement on the body portion, securing the measurement device to the body portion while taking a measurement on the body portion and/or using the measurement device to obtain an angular measurement related to the body portion.

### A BRIEF DESCRIPTION OF THE DRAWINGS

FIG. 1 is a prospective view of an embodiment of a measurement device according to the present invention.

FIGS. 1A and 1B are top down views of alternative anatomically shaped end embodiments.

FIGS. 1C, 1D and 1E are section A-A views of alternative contours of an embodiment of the anatomically shaped end.

FIGS. 1F, 1G and 1H illustrate different measurement devices of different sizes.

FIG. 1I illustrates an embodiment of a measurement device of the present invention positioned adjacent the superior aspect of the patella.

FIG. 1J illustrates an embodiment of a measurement device of the present invention positioned adjacent the popliteal fossa.

FIG. 1K illustrates an embodiment of a measurement device of the present invention positioned adjacent the anticubutal fossa.

FIG. 2 is an alternative embodiment of the measurement device of FIG. 1.

FIG. 3 is another alternative embodiment of a measurement device according to the present invention.

FIG. 4 is a prospective view of an alternative embodiment of the measurement device of FIG. 3.

FIG. 5 is an alternative embodiment of a measurement device of the present invention including fixed linear measurement devices.

FIG. 6 is a prospective view of a measurement device of the present invention having a slidable linear measurement device.

FIG. 7A is a prospective view of the measurement device of FIG. 6 with securing straps.

FIG. 7B is an alternative embodiment of the measurement device of FIG. 7A with an alternative strap embodiment.

FIGS. 8, 9, 10, 11, 12A and 12B are improved goniometers including embodiments of the measurement device of the present invention.

### DETAILED DESCRIPTION OF THE INVENTION

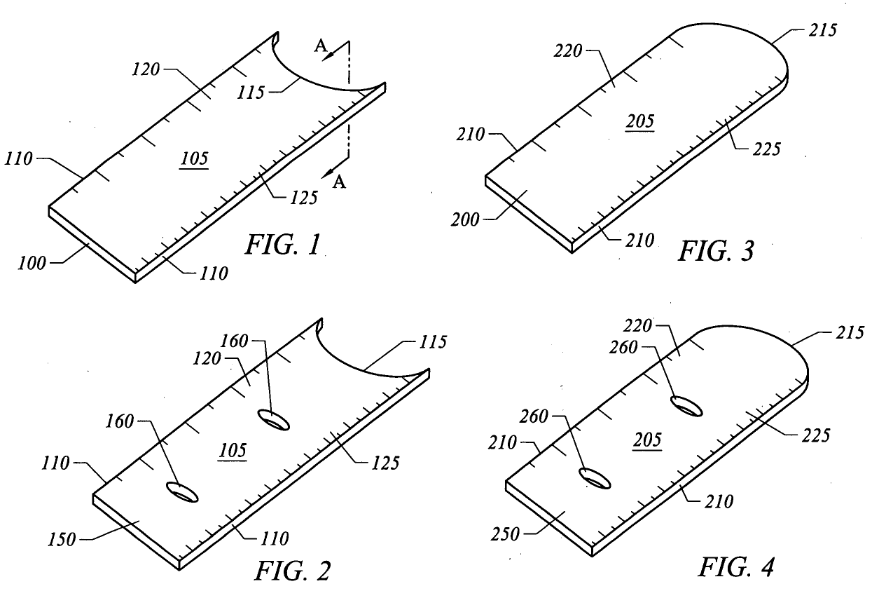

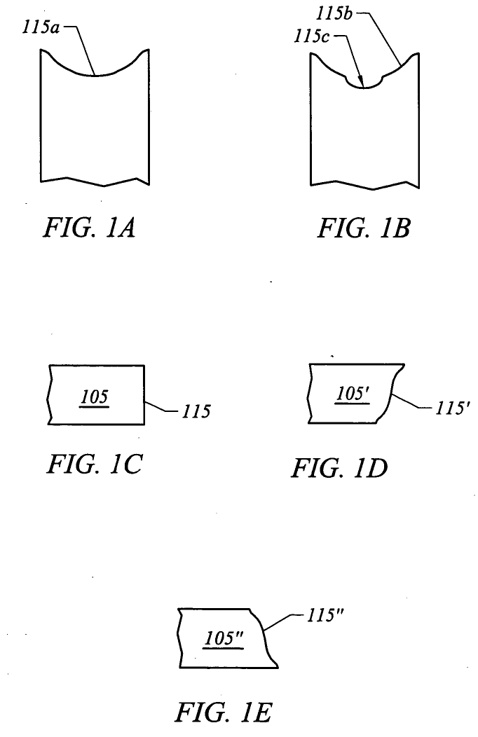

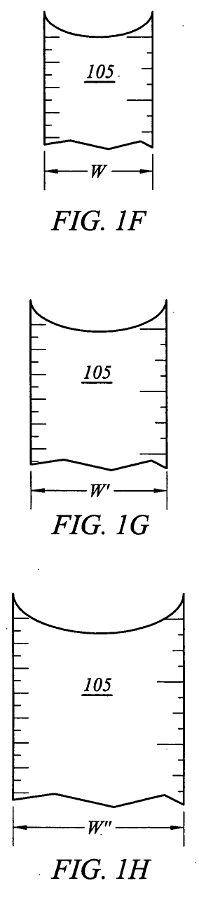

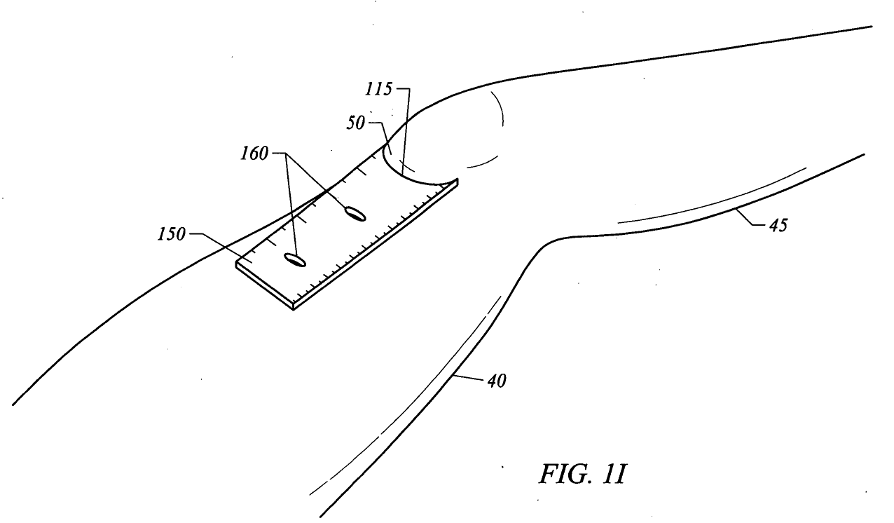

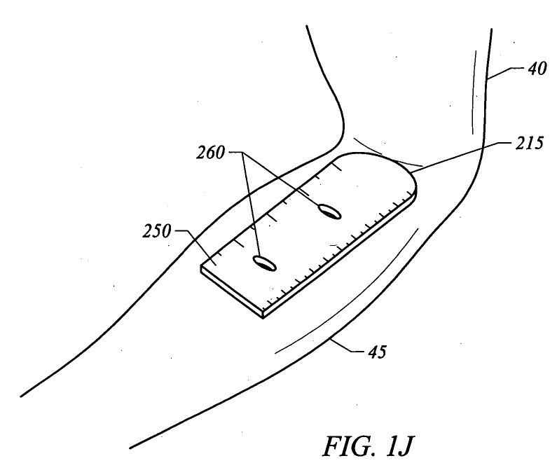

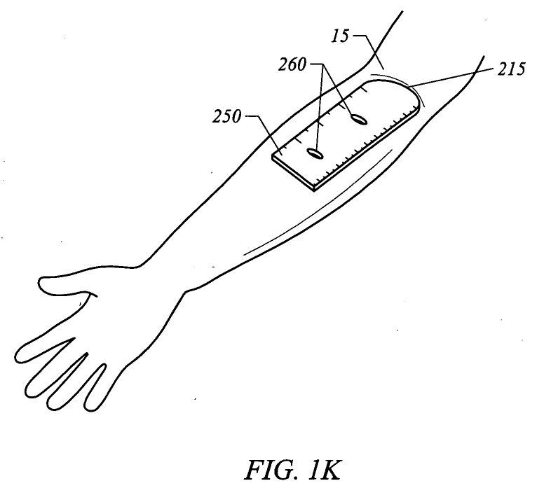

Measuring device 100, illustrated in FIG. 1, is one embodiment of the present invention. Measuring device 100 includes a generally rectangular base 105 having elongated sides 110 and an anatomically shaped end 115. The anatomically shaped end 115 is configured to engage with a musculoskeletal anatomical landmark on a body. A plurality of measurement increments indicates distance from the anatomically shaped end 115. In one aspect, the plurality of measurement increments correspond to an accepted measurement system. The English/US measurement system (inches and fractions thereof) and the metric system are both examples of accepted measurement systems. In the illustrative example, inches and fractions thereof 120 and centimeters and fractions thereof 125 from the anatomically shaped end 115 are both indicated in the illustrative embodiments. The measuring device 105 may be formed from virtually any rigid, durable material, such as, for example, plastic. In one preferred embodiment, the measuring device 105 is formed from a transparent hard plastic colored with a highly visible color for ease of locating. Examples of highly visible colors include orange, lime green and yellow.

The anatomically shaped end 115 provides particular advantage and represents an improvement over conventional measurement devices. When the measurement device 100 is used for measurements, it is placed against a portion of a body, a limb for example, to be measured. The unique contours and shape of the anatomically shaped end 115 are advantageously used to engage with a musculoskeletal anatomical landmark useful in measuring a dimension of the limb or portion thereof. In many instances, a musculoskeletal anatomical landmark exists in proximity to the limb that can be beneficially utilized to increase biomechanical measurement reliability, repeatability and accuracy. Accordingly, the desired anatomical measurement determines what musculoskeletal anatomical landmark to use. The shape and contour of the musculoskeletal anatomical landmark determines the optimum and cooperative shape and contour for the anatomically shaped end 115. As a result, the overall dimensions of measurement devices of the invention and the shape of the anatomically shaped end 115 can vary from the illustrative embodiment of FIG. 1. Any of a variety of shapes may be used to engage with any of a wide variety of musculoskeletal anatomical landmarks on mammals, both human and non-human.

The shape of the anatomically shaped end refers to the appearance of the anatomically shaped end when viewed from the top of the measuring device. For example, the anatomically shaped end 115 (FIG. 1) has a concave shape, while the anatomically shaped end 215 (FIG. 2) has a convex shape. FIG. 1A and FIG. 1B illustrate still other possible shapes. FIG. 1A illustrates a generally elliptical shape 115A. FIG. 1B illustrates a complex curvilinear shape having a generally parabolic shape 115B superimposed with an additional curvilinear feature 115C. The additional curvilinear feature 115C can be configured for placement against a particular musculoskeletal anatomical feature. For example, when a measurement of the forearm is desired the generally parabolic shape 115B may be configured to align with the anticubital fossa; the additional feature 115C may be configured to align with an expected or likely position of another anatomical feature.

The contour of the anatomically shaped end refers to the cross sectional shape presented by the anatomically shaped end. An anatomically shaped end may have a flat contour as illustrated in FIG. 1C. FIG. 1C is a section A-A view of device 105 of FIG. 1. More complex contours are possible, such as for example, contour 115′ (section A-A of a device 105′ in FIG. 1D) and contour 115″ (section A-A of a device 105″ in FIG. 1E) illustrate compound curvilinear contour styles. Virtually any contour style may be used to conform with a musculoskeletal feature of interest.

In addition to the dimensional variations for the anatomically shaped end described above, the overall dimensions of measurement device embodiments of the present invention may also be adjusted to accommodate different sizes of musculoskeletal anatomical landmarks. Size refers to the difference in musculoskeletal anatomical features between, for example, males and females or adults and children. FIGS. 1F-1H illustrate how measuring device width can vary (W<W′<W″). Again, consider the case where the body is a human and the musculoskeletal anatomical landmark is the patella. The size of the patella will vary depending upon the gender and age of the person being measured. Accordingly, there are embodiments of the present invention having an anatomically shaped end of varying contour and cross-section to accommodate patellas of various sizes and shapes. In the illustrated example in FIG. 1F, the device 105 has a width W suited for the measurement of infants. In FIG. 1G, the device 105 has a width W′ suited, for example, for the measurement of adult female patellas. In FIG. 1H, the device 105 has a width W″ suited to the measurement of adult male patellas. While described with respect to the patella, numerous alternative embodiments exist for a number of other useful musculoskeletal anatomical landmarks. In addition, using the principles described above, embodiments of the present invention can accommodate the musculoskeletal anatomical landmarks in non-human bodies, such as for example, domesticated animals and other creatures treated or of interest by veterinarians or studied by scientists.

Examples of musculoskeletal anatomical landmarks in humans include, but are not limited to, the patella, the anticubital fossa and the popliteal fossa. FIG. 1I illustrates a measuring device 150 positioned on the upper thigh 40 with the anatomically shaped end 115 aligned with the superior aspect of the patella 50. Note how the shape of the anatomically shaped end 115 ensures proper measurement device placement by conforming with the exterior shape of the patella's superior aspect. FIG. 1J illustrates a measuring device 250 positioned on the calf 45 with the anatomically shaped end 215 aligned against the popliteal fossa. Note how the shape of the anatomically shaped end 215 ensures proper placement by conforming with the shape of the popliteal fossa. FIG. 1K illustrates a measuring device 250 positioned on the forearm with the anatomically shaped end 215 aligned against the anticubital fossa 15. Note how the shape of the anatomically shaped end 215 ensures proper placement by conforming with the shape of the anticubital fossa 15.

Measuring devices according to embodiments of the present invention have particular utility in the orthopedic field. When evaluating the disability or atrophy of a muscular group on the body, conventional measurement devices fail to provide reliable and consistent measurements between different measurements on the same patient and the measurement of the same anatomy on different patients. The variability in measurements, both within the same patient and within a large pool of patients, leads to undesired variability when trends in disability are attempted. One representative application for device 100 includes comparative measurements of the vastus lateralis and the quadriceps femoris when taken at spacing of 2 inches and 4 inches from the superior aspect of the patella where the quadriceps attach to the patella. For example, the American Medical Association guidelines for evaluating disability looks to the measurements at 2 inches and 4 inches above the superior aspect of the patella to evaluate disability. The ability to accurately, reliably and repeatedly obtain these measurements is critical to correctly assessing the state of atrophy or improvement in the state of the muscle group measured. Further details regarding the evaluation of disabilities can be found in “Guides to the Evaluation of Permanent Impairment,” 5^{th} Ed., by Cochiarella and Anderson.

In order to take the above-mentioned measurement, the measurement device 100 is placed on the bare skin of the patient's leg (see FIG. 1I). The anatomically shaped end 115 is aligned with and pressed against the superior aspect of the patella. The user then marks the skin of the patient at the desired measurement points. In the illustrated embodiment, the device 150 has apertures 160 that are positioned 2 inches and 4 inches above the anatomically shaped end 115. The user inserts a marking device into the apertures 160 to mark the skin. Next, the device 150 is removed and the circumference of the thigh is measured at the above-made marks using a flexible linear measurement device, such as a retractable steel measuring tape. The inventive device provides repeatable and more reliable measurements when compared to conventional measurement devices because, among other reasons, the rigid structure of the base and the anatomically shaped end ensure more accurate and repeatable measurement device placement during later measurements on the same patient. Additionally, the advantageous shape of measurement devices of the present invention standardizes the measurement instrument placement to provide more reliable measurement data for comparison between different patients.

FIG. 2 illustrates measurement device 150 which is an alternative embodiment of the present invention. Measurement device 150 is similar to measurement device 100 with the addition of apertures 160 formed with the base 105. Measurement device 150 is used in a similar fashion to measurement device 100 with the exception that instead of marking the patient's leg along the elongated sides 110, the anatomy to be measured is marked using the apertures 160 to create the measurement reference marks. While the apertures 160 are illustrated as elliptical, it is to be appreciated that a wide variety of different aperture shapes could be used. For example, apertures 160 may be rectangular, circular, curvilinear, or any other shape that allows a user to mark the skin beneath the measurement device.

Measuring device 200 is illustrated in FIG. 3. Measuring device 200 is an alternative embodiment of a measuring device of the present invention. The measuring device 200 is similar to the measuring device 100 of FIG. 1 in that when in use the reference marks are made adjacent the elongated edges 225. However, the measurement device 200 differs from measurement devices 100, 150 in that the anatomically shaped end 215 has a convex shape as opposed to the concave shape of anatomically shaped end 115. Anatomically shaped end 215 has the advantage of working with musculoskeletal anatomical landmarks that are more readily utilized by this convex shape. As an illustrative example, the measurement device 200 would be useful when measuring the calf because the anatomically shaped end 215 will readily engage with and conform to the popliteal fossa, thereby greatly increasing the reliability, repeatability and accuracy of calf measurements (See discussion of FIG. 1J above).

Measurement device 250 is illustrated in FIG. 4. Measurement device 250 represents another alternative embodiment of a measurement device of the present invention. Measurement device 250 is similar to measurement device 200 described above in FIG. 3 in that the anatomically shaped end 215 is convex. However, in use, the measurement device 250 is similar to measurement device 150 (FIG. 2). Measurement device 250, like measurement device 150, includes apertures for ease of marking the underlying skin for more accurate measurements. Measurement device 250 includes apertures 260. While both measurement device 250 and measurement device 150 illustrate two apertures, it is to be appreciated that a plurality of apertures may be utilized or only a single aperture may be utilized. Additionally, the apertures may be spaced at regular or irregular intervals from the anatomically shaped end.

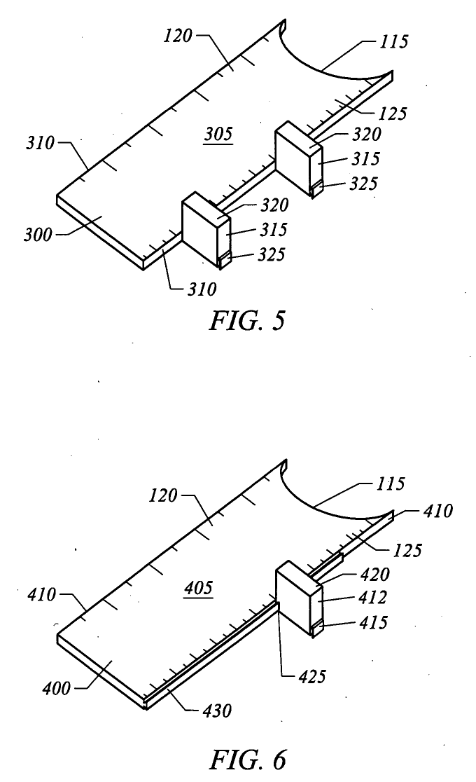

FIGS. 5 and 6 illustrate embodiments of the measuring device of the present invention where the measuring device remains in place during measurement of the anatomy of interest. Measurement device 300 is illustrated in FIG. 5 and is similar to measurement device 100 of FIG. 1. Measurement device 300 includes a base 305 having elongated sides 310. Elongated sides 310 have been modified to accept a linear measurement device 310. The linear measurement device 310 includes a casing 320 and a tab 315. Tab 315 is used to extend a flexible linear measurement device contained within casing 320. The casing 320 is coupled to an elongated side 310 of base 305. The casing 320 could be glued, bonded, joined or otherwise affixed to the elongated side 310. Alternatively, the base 305 and casing 320 could be integrally formed from a single piece of material, such as, for example, plastic, Plexiglas, or other rigid and durable material.

One advantage of measuring device 300 is that it remains in place during the measurement of the anatomy of interest, thereby increasing the measurement accuracy. In use, the anatomically shaped end 115 of measurement device 300 is placed against a musculoskeletal anatomical landmark. Next, the tabs 315 are grasped, and the flexible linear measurement device is extended about the circumference of the anatomy to be measured, thereby holding the device in place.

Measurement device 400, another alternative embodiment of a measuring device of the present invention, is illustrated in FIG. 6. Measurement device 400 includes a base 405, elongated sides 410, and an anatomically shaped end 115. Additionally, a railing 430 is coupled to an elongated side 410. The railing 430 could be glued, bonded, joined, or otherwise afixed to the elongated side 410. Alternatively, the railing 430 and the base 405 could be formed from a single piece of material, such as, for example, plastic, Plexiglas, or other rigid material.

Railing 430 includes a cross-sectional shape that operates as a mating feature. As illustrated, the cross-sectional shape 430 is trapezoidal. The linear measurement device casing 420 includes a mating feature 425 that is complementary to the mating shape of the railing 430. In this illustrative example, the casing mating feature 425 is trapezoidal and mates cooperatively with the trapezoidal shape of the railing 430 in a dovetail fashion. While the illustrated example includes a mating feature that has a trapezoidal cross-section, it is to be appreciated that other shapes may be used to obtain the slidable relationship between the measurement device casing 420 and the railing 430.

One advantage of the measuring device 400 is that a single measurement device may be used to obtain multiple measurements while the device 400 remains against the anatomy. A user may position the measurement device 410 at a first position, extend the tab 415 and attach the measurement device about the circumference of the anatomy to be measured to obtain a first measurement. Next, the user retracts the tab 415 and measuring tape back into the casing 420 and indexes the casing to a different location without disturbing the position of the measurement device 400 against the anatomy. This process repeats as desired to obtain a second or even more measurements while the measurement device 400 is advantageously placed with the anatomically shaped end 115 positioned against a musculoskeletal anatomical landmark on a body.

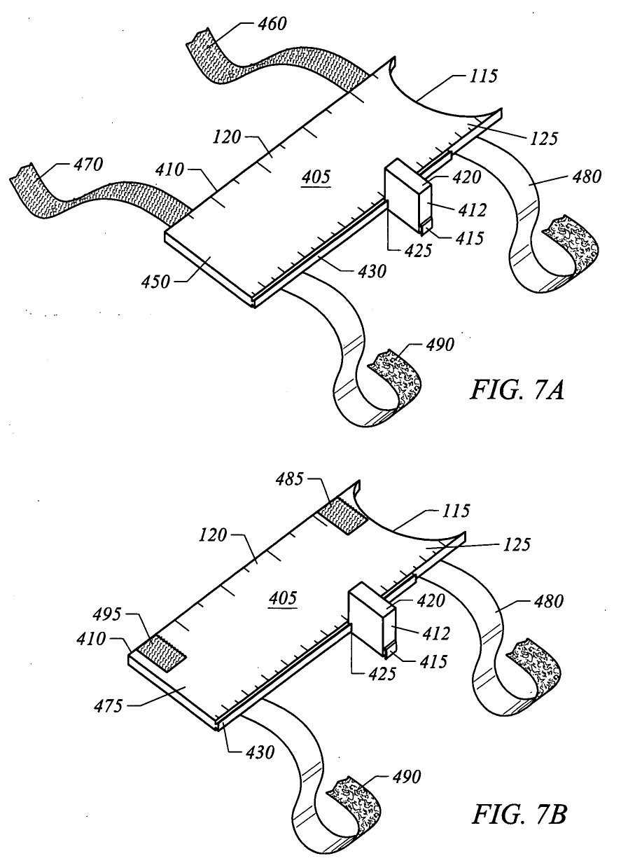

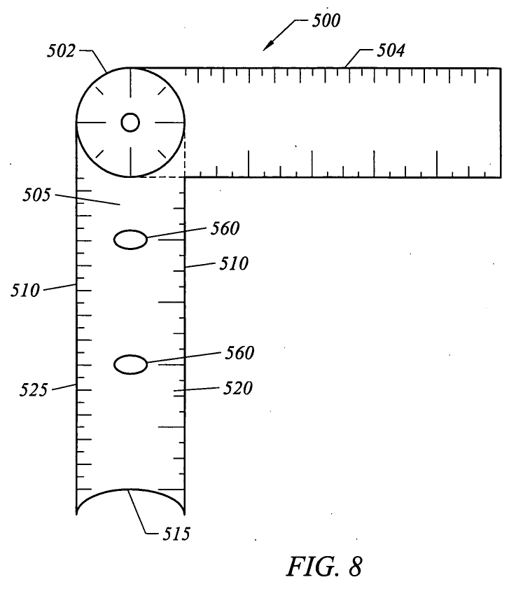

Measurement devices 450, 475 are illustrated in FIGS. 7A and 7B. Measurement devices 450, 475 are alternative embodiments of the measurement devices of the present invention that include securing means to maintain the measurement device in position on the anatomy of interest while measurements are taken. Both measurement device 450 and measurement device 475 illustrate the use of Velcro straps to secure the measurement device against the anatomy of interest. Measurement device 450 uses two pairs of Velcro straps 480, 460 and 490, 470. In use, the user wraps Velcro straps 460, 470 about the anatomy, and Velcro straps 480 and 490 are then placed over straps 460 and 470 to secure the measurement device 450 to the anatomy of interest. Measurement device 475 in FIG. 7B uses a pair of Velcro straps 480, 490 that wrap around the anatomy to secure to complementary Velcro pieces 485, 495 on base 405. One advantage of securing the measurement device to the anatomy is that the user then has both hands free to index the measurement device 410 and also extend and retract the linear measurement device using tab 415. These advantages simplify the measurement process and shorten the amount of time taken to obtain accurate, reliable and repeatable anatomical measurements.

The advantages of embodiments of the present invention may also be used to improve the accuracy of measurements taken by conventional goniometers. Improved goniometers 500, 550 and 575 will now be discussed in turn. Goniometer 500 (FIG. 8) includes a hub 502 and arms 504, 505. Arms 504, 505 are rotatably coupled to hub 502. Arm 505 is of a similar design and configuration and provides the advantages of measurement device 150 in FIG. 2. Arm 505 has a generally rectangular shape with elongated sides 510. Arm 505 also includes apertures 560 and an anatomically shaped end 515.

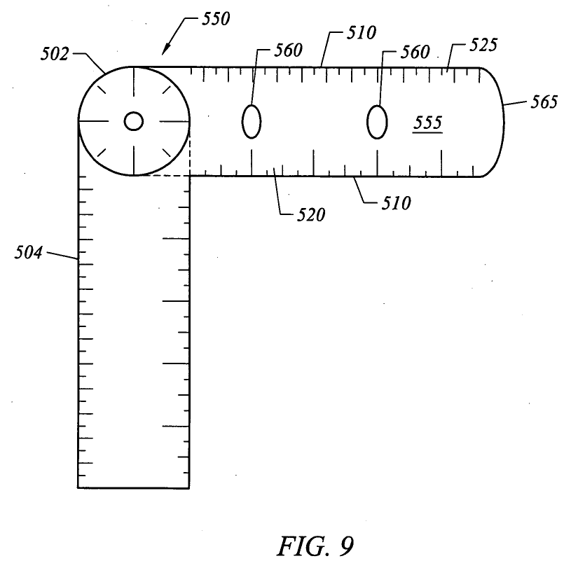

Improved goniometer 550 is illustrated in FIG. 9. Goniometer 550 includes a hub 502 and arms 505, 555 that are rotatably coupled to the hub 502. Arm 555 is similar in design, configuration use and provides the same advantages as device 250 of FIG. 4. Arm 555 has a generally rectangular shape with elongated sides 510 and apertures 560. Like measurement device 250, the anatomically shaped end 565 of improved goniometer 550 is convex.

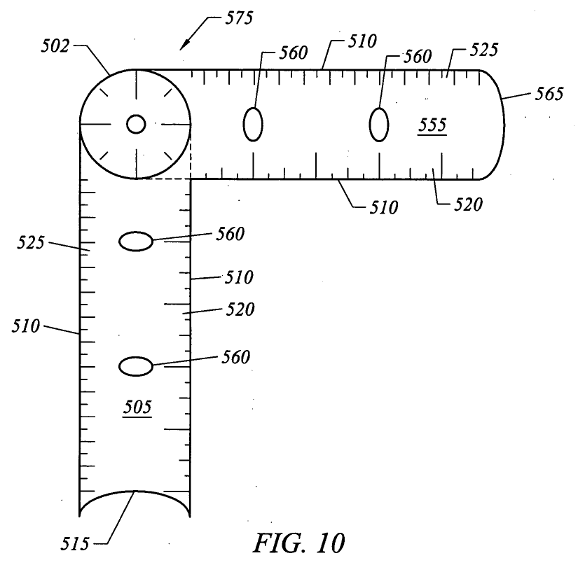

Improved goniometer 575 is illustrated in FIG. 10. Improved goniometer 575 includes a hub 502 and arms 505, 555 that are the same as described with regard to FIGS. 8 and 9 and improved goniometers 500, 550.

Each of the improved goniometers described above provides the additional advantages of the measurement devices of the present invention to a widely used and readily available clinical measurement device. The embodiments of the improved goniometers described above will simplify clinical measurements and enable more accurate and reliable measurements to be taken. Improved goniometer 575 in particular provides the added advantage of having both a convex and concave anatomically shaped end. By having two types of anatomically shaped ends, improved goniometer 575 may more readily and easily be applied to a wide variety, perhaps virtually any, measurement situation likely to arise in a clinical setting. Because the improved goniometers above provide additional measurement capabilities, users may reduce the number of measurement devices needed in a clinical setting.

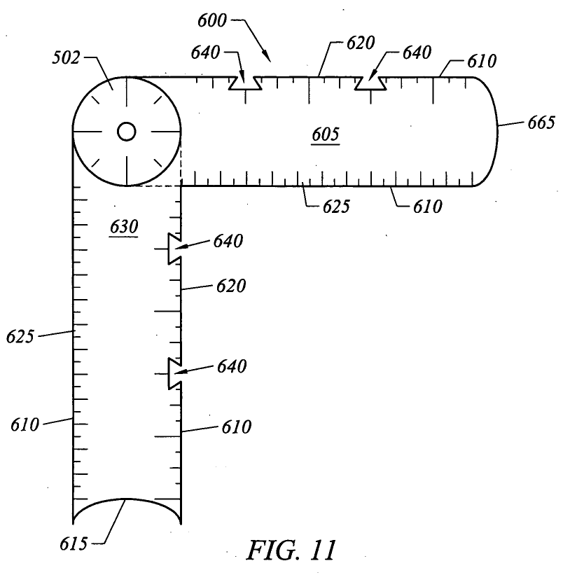

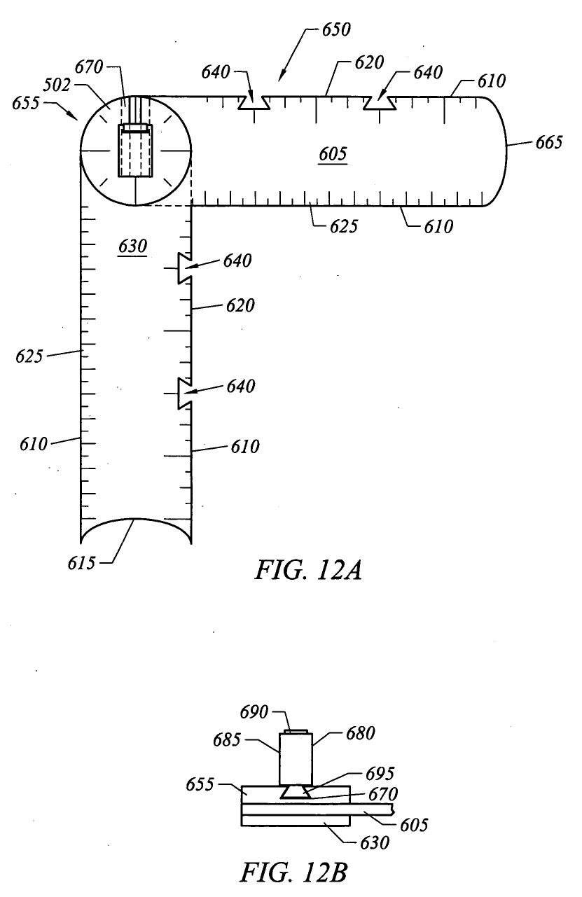

Turning now to FIGS. 11 and 12, improved goniometers 600 and 650 will be described in turn. Improved goniometer 600 (FIG. 11) includes a hub 502 and arms 605, 630 having generally rectangular shapes including elongated sides 610 with anatomically shaped ends. Arm 605 includes a convex anatomically shaped end 665 while arm 630 includes a concave anatomically shaped end 615. Advantageously, arms 605, 630 include mating features 640 along one elongated side 610. Mating feature 640 are positioned at desired distances from the anatomically shaped ends to facilitate measurements.

In use, a measurement device casing having a complementary mating feature is placed within and secured by the mating feature 640. The cooperative mating of the arm to a linear measurement device case is similar to the discussion above regarding railing 430 and mating feature 425 (FIG. 6). The linear measurement device casing is secured to the goniometer 600 via engagement with mating feature 640. This engagement is firm enough that extension and retraction operations of the linear measurement device does not disturb the coupling between the measurement device casing and the attached arm.

FIGS. 12A and 12B illustrate an embodiment of an improved goniometer 650 that provides onboard storage of a linear measurement device 680. Improved goniometer 650 is similar to goniometer 600 described above with the exception of the modified hub 655. The modified hub 655 includes a shaped recess 670 that is shaped to mate with shaped feature 675 of the measurement device 680. Linear measurement device 680 has a casing 685 and a pull tab 690. The casing 685 includes a shaped feature 695 that engages with the shaped recess 670. For simplicity, the shaped mating features 670, 640 are similarly shaped. In this way, a single shaped feature 695 may be used to secure the linear measurement device 680 to an arm 605, 630 during measurement or the hub 655 for storage. The linear measurement device 680 is secured to an arm 605, 630 by urging the shaped feature 695 into a mating feature 640. The linear measurement device 680 may be fastened to the hub 655 when not in use by securing shaped feature 695 into mating recess 670. Thus, the improved goniometer 650 provides the advantage of a fully functioning goniometer as well as the advantages of the measurement devices of the present invention, namely, more accurate, reliable and repeatable measurements. In alternative embodiments, goniometers 600, 650 may also include one or more apertures 560 as illustrated and described herein.

Embodiments of the present invention also include methods for obtaining a measurement on a body portion. In one aspect, the method includes placing a measurement device having an elongate side and an anatomically shaped end adjacent the body portion to be measured so that the anatomically shaped end engages a musculoskeletal anatomical landmark adjacent the body portion to be measured. Next, use the measurement increments on the elongate side of the measurement device to determine a position for taking a measurement on the body portion relative to the musculoskeletal anatomical landmark engaged by the anatomically shaped end.

In another aspect, embodiments of the measurement method may include taking a measurement of the body portion at the determined position for taking a measurement on the body portion. The measurement taken may include any type of measurement related to the body portion. In one specific embodiment, the measurement taken is the circumference of the body portion. As discussed herein, the method may also be practiced by placing the anatomically shaped end against the musculoskeletal anatomical landmark adjacent the body portion to be measured. In additional aspects, the anatomical landmark may be the patella, the anticubital fossa and the popliteal fossa. The method of making the measurement may also include marking the body portion at the determined position for taking a measurement. In one aspect, marking the body portion at the determined position is performed using an aperture formed in the measurement device. In another aspect, the measurement is performed by attaching another measurement device to the elongate side at the determined position. In another aspect, the method includes securing the measurement device to the body portion while taking a measurement on the body portion. In another aspect, the method includes using the measurement device to obtain an angular measurement related to the body portion.

While the invention has been described in connection with specific embodiments, it will be understood that embodiments of the invention are capable of further modification. For example, the described measurement device embodiments refer to taking circumferential measurements of the anatomy. It is to be appreciated that embodiments of the measurement device of the present invention may be used for other measurements as well. This application is intended to cover any variations, uses, or adaptations of the invention following, in general, the principles of the invention and including such departures from the present disclosure as come within known or are the customary practice within the art to which the invention pertains.

1. A measurement device comprising:
a base having a rectangular shape including elongated sides and an anatomically shaped end configured to engage with a musculoskeletal anatomical landmark on a body; and
a plurality of measurement increments spaced along an elongated side to indicate distance from the anatomically shaped end.

2. The measurement apparatus according to claim 1 wherein the anatomically shaped end is configured to engage with a musculoskeletal anatomical landmark selected from the group consisting of the patella, the anticubital fossa and the popliteal fossa.

3. The measurement apparatus according to claim 1 wherein the anatomically shaped end is convex or concave.

4. The measurement apparatus according to claim 1 wherein a plurality of measurement increments spaced along an elongated side to indicate distance from the anatomically shaped end comprises a plurality of measurement increments spaced along one elongated side to indicate inches and fractions thereof from the anatomically shaped end and a plurality of measurement increments indicates along another elongated side centimeters and fractions thereof from the anatomically shaped end.

5. The measurement device of claim 1 wherein the base comprises a goniometer arm.

6. A measurement device comprising:
a base having an elongated side and an anatomically shaped end configured to engage with a musculoskeletal anatomical landmark on a body;
a plurality of measurement increments spaced along the elongated side to indicate distance from the anatomically shaped end; and
an aperture formed within the base at a predetermined measurement increment from the anatomically shaped end.

7. The measurement apparatus according to claim 6 wherein the anatomically shaped end is configured to engage with a musculoskeletal anatomical landmark selected from the group consisting of the patella, the anticubital fossa and the popliteal fossa.

8. The measurement apparatus according to claim 7 wherein the predetermined measurement increment from the anatomically shaped end is at either 2 inches or 4 inches.

9. The measurement apparatus according to claim 7 further comprising a second aperture formed within the base at a predetermined measurement increment from the anatomically shaped end.

10. A measurement device comprising:
a base having an elongated side and an anatomically shaped end configured to engage with a musculoskeletal anatomical landmark on a body;
a plurality of measurement increments spaced along the elongated side to indicate distance from the anatomically shaped end;
at least one aperture formed within the base at a predetermined measurement increment from the anatomically shaped end; and
a measurement device removably coupled to the base.

11. The measurement device according to claim 10 wherein the measurement device comprises a tape measurer.

12. The measurement device according to claim 10 wherein the base includes a mating feature and the measurement device includes a mating feature that complements the base mating feature so that the measurement device is removably coupled to the base by engaging the mating features.

13. The measurement device according to claim 12 wherein the base mating feature is positioned on the elongate side.

14. The measurement device according to claim 10 further comprising a plurality of apertures formed in the base and the measurement device is in slideable relation to the base so that the measurement device may be indexed along the base and aligned to an aperture.

15. The measurement device according to claim 10 further comprising at least one strap to secure the base in a desired relationship to a musculoskeletal anatomical landmark on a body.

16. A goniometer comprising:
a hub;
an arm coupled to the hub, the arm having a rectangular shape including elongated sides and an anatomically shaped end configured to engage with a musculoskeletal anatomical landmark on a body; and
a plurality of measurement increments spaced along an elongated side of the arm to indicate distance from the anatomically shaped end.

17. A goniometer according to claim 16 further comprising a second arm coupled to the hub, the second arm having a rectangular shape including elongated sides and an anatomically shaped end configured to engage with a musculoskeletal anatomical landmark on a body; and
a plurality of measurement increments spaced along an elongated side of the second arm to indicate distance from the anatomically shaped end.

18. A goniometer according to claim 17 wherein at least one of the anatomically shaped end of the first arm or the anatomically shaped end of the second arm is configured to engage with a musculoskeletal anatomical landmark selected from the group consisting of: the patella, the anticubital fossa and the popliteal fossa.

19. A goniometer according to claim 17 wherein the anatomically shaped end of one of the first arm or the second arm is convex and the anatomically shaped end of other of the first arm or the second arm is concave.

20. A goniometer according to claim 17 wherein at least one mating feature is located on either the arm or the second arm.

21. A goniometer according to claim 16 further comprising at least one mating feature located on the hub.

22. A goniometer according to claim 21 further comprising a measurement device casing having a mating feature that cooperatively joins with the at least one mating feature located on the hub to releasably secure the measurement device to the hub.

23. A goniometer according to claim 22 further comprising a measurement device casing having a mating feature that cooperatively joins with at least one mating feature located on either the arm or the second arm to releasably secure the measurement device to either the arm or the second arm.

24. A method for obtaining a measurement on a body portion, comprising:
placing a measurement device having an elongate side and an anatomically shaped end adjacent the body portion to be measured so that the anatomically shaped end engages a musculoskeletal anatomical landmark adjacent the body portion to be measured; and
using measurement increments on the elongate side of the measurement device to determine a position for taking a measurement on the body portion relative to the musculoskeletal anatomical landmark engaged by the anatomically shaped end.

25. The method according to claim 24 further comprising taking a measurement of the body portion at the determined position for taking a measurement on the body portion.

26. The method according to claim 25 wherein the measurement comprises a circumference of the body portion.

27. The method according to claim 24 wherein the musculoskeletal anatomical landmark adjacent the body portion to be measured is selected from the set consisting of: the patella, the anticubital fossa and the popliteal fossa.

28. The method according to claim 24 further comprising marking the body portion at the determined position for taking a measurement.

29. The method according to claim 28 wherein marking the body portion at the determined position is performed using an aperture formed in the measurement device.

30. The method according to claim 24 further comprising attaching another measurement device to the elongate side at the determined position for taking a measurement on the body portion.

31. The method according to claim 25 further comprising securing the measurement device to the body portion while taking a measurement on the body portion.

32. The method according to claim 24 further comprising using the measurement device to obtain an angular measurement related to the body portion.
